(19) United States
(12) Patent Application Publication
(10) Pub. No.: US 2018/0168844 A1
(43) Pub. Date: Jun. 21, 2018

(54) Thumb Rehabilitation Splinting Device

(71) Applicant: Jeff Garcia, Lakewood, CO (US)

(72) Inventor: Jeff Garcia, Lakewood, CO (US)

(21) Appl. No.: 15/387,086

(22) Filed: Dec. 21, 2016

(51) Int. Cl. A61F 5/058; A61F 5/01

(52) U.S. Cl. CPC A61F 5/05866; A61F 5/0118

(57) ABSTRACT

A thumb rehabilitation splinting device for selectively immobilizing a thumb includes a sleeve that has a first end and a second end, which are open. The sleeve is substantially complementary to a palm and a forearm of a user. A hole, configured to insert a thumb to position the thumb external to the sleeve, is positioned in the sleeve proximate to the first end. A tube is configured to insert at least a portion of a distal phalanx of the thumb. First and second couplers that are coupled to the tube are selectively couplable to a side and a palm face of the sleeve, respectively. A slat, which is rigid, is coupled to the palm face and is configured to substantially immobilize a wrist. The first and second couplers are configured to adjustably substantially immobilize the thumb relative the hand of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art including information disclosed under 37 CFR 1.97 and 1.98

The disclosure and prior art relates to splinting devices and more particularly pertains to a new splinting device for selectively immobilizing a thumb.

### BRIEF SUMMARY OF THE INVENTION

An embodiment of the disclosure meets the needs presented above by generally comprising a sleeve that has a first end and a second end, which are open. The sleeve is substantially complementary to a palm and a forearm of a user. A hole, configured to insert a thumb to position the thumb external to the sleeve, is positioned in the sleeve proximate to the first end. A tube is configured to insert at least a portion of a distal phalanx of the thumb. First and second couplers that are coupled to the tube are selectively couplable to a side and a palm face of the sleeve, respectively. A slat, which is rigid, is coupled to the palm face and is configured to substantially immobilize a wrist. The first and second couplers are configured to adjustably substantially immobilize the thumb relative the hand of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a thumb rehabilitation splinting device according to an embodiment of the disclosure.

FIG. 2 is a top view of an embodiment of the disclosure.

FIG. 3 is a bottom view of an embodiment of the disclosure.

FIG. 4 is an in-use view of an embodiment of the disclosure.

FIG. 5 is an in-use view of an embodiment of the disclosure.

### DETAILED DESCRIPTION OF THE INVENTION

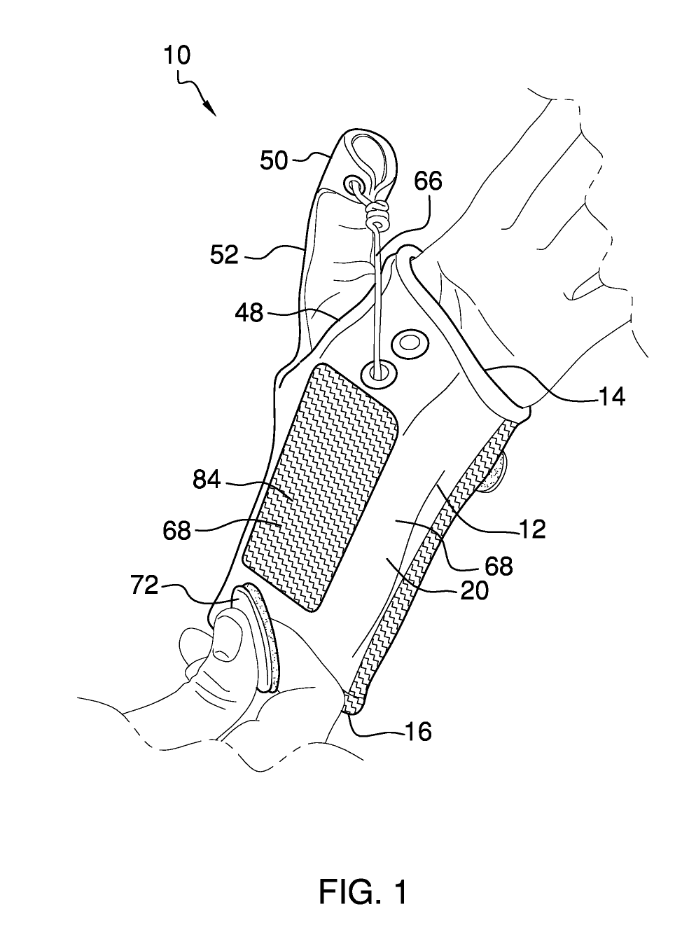

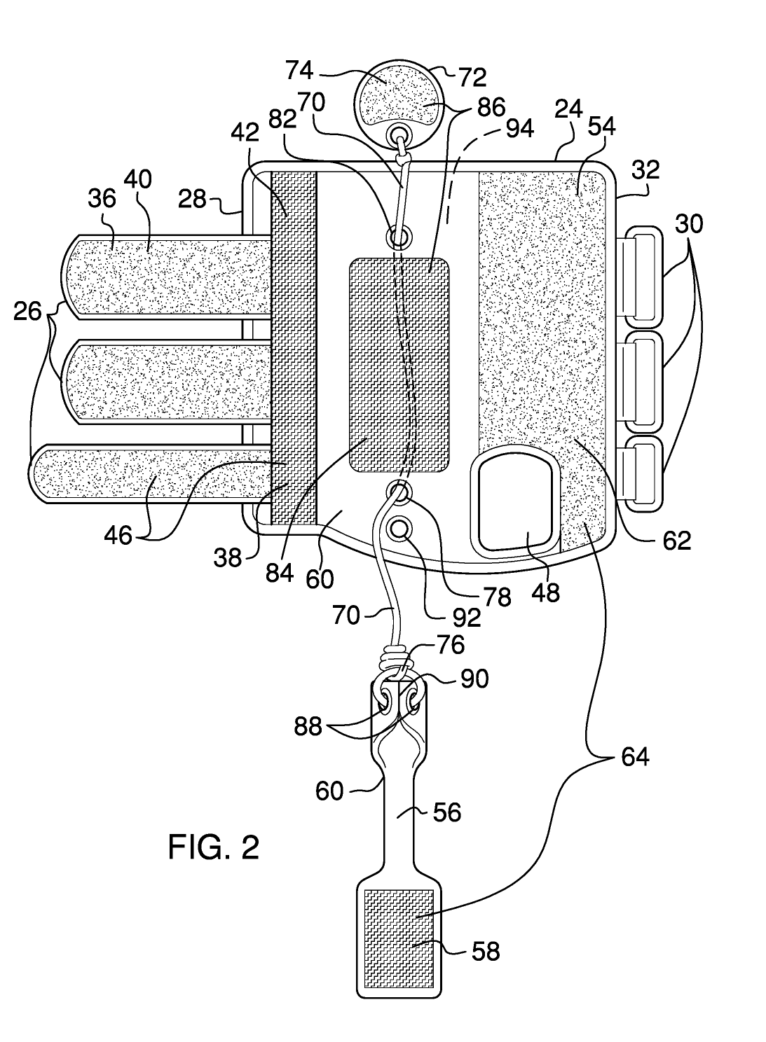

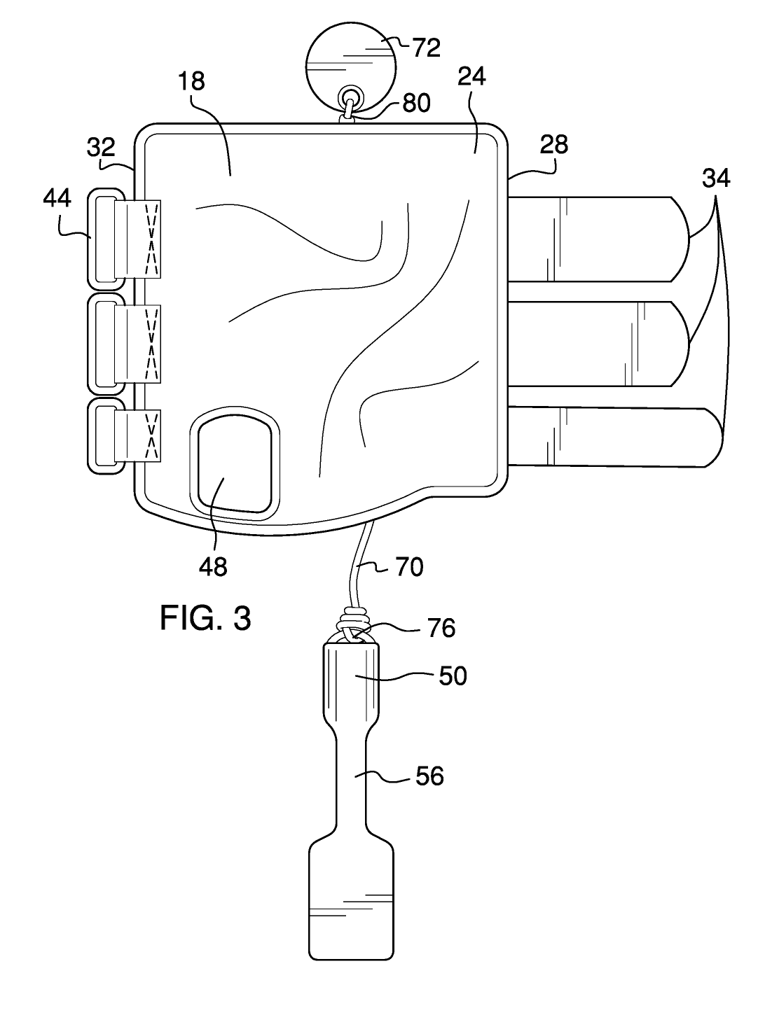

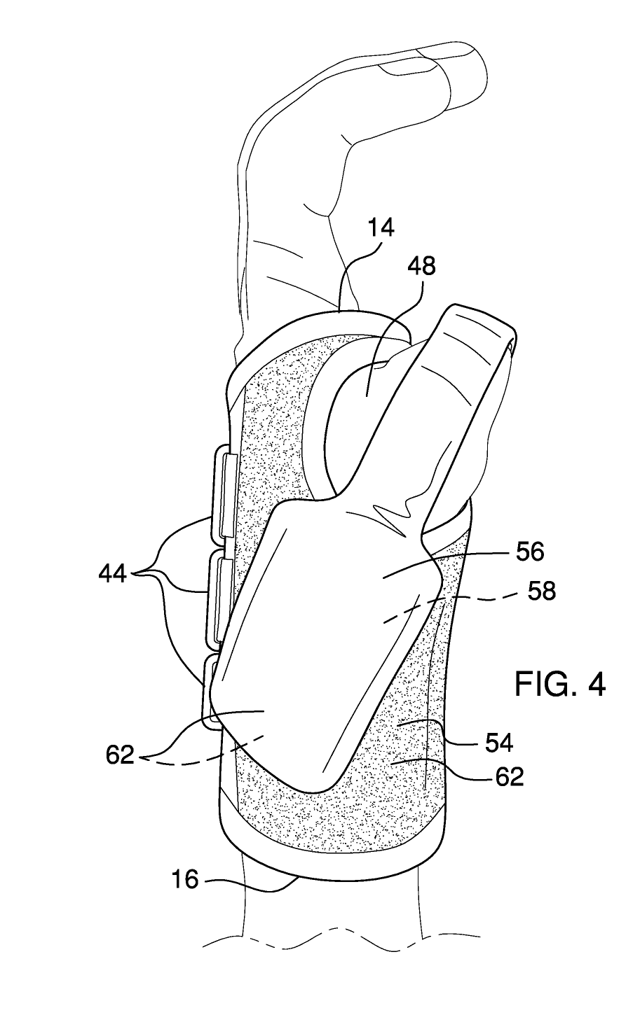

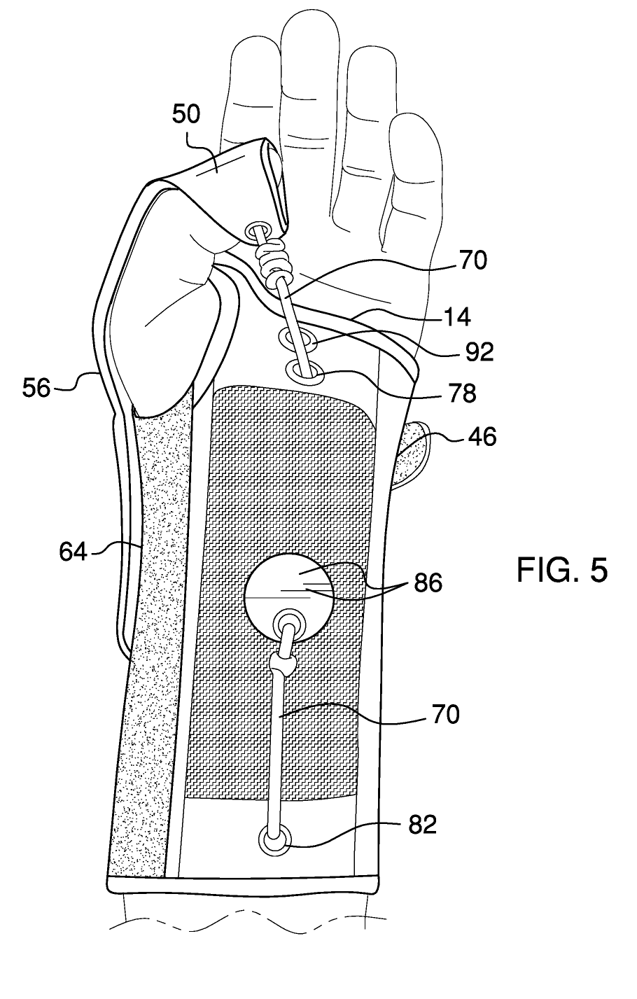

With reference now to the drawings, and in particular to FIGS. 1 through 5 thereof, a new splinting device embodying the principles and concepts of an embodiment of the disclosure and generally designated by the reference numeral 10 will be described.

As best illustrated in FIGS. 1 through 5, the thumb rehabilitation splinting device 10 generally comprises a sleeve 12 that has a first end 14 and a second end 16. The first end 14 and the second end 16 are open. The sleeve 12 is substantially complementary to a palm and a forearm of a user. In one embodiment, the sleeve 12 comprises an inner layer 18 and an outer layer 20. In another embodiment, a slit 22 is positioned in the sleeve 12. The slit 22 extends from the first end 14 to the second end 16. The sleeve 12 is selectively configurable as a panel 24.

A first connector 26 is coupled to a first edge 28 of the panel 24. A second connector 30 is coupled to a second edge 32 of the panel 24. The second connector 30 is complementary to the first connector 26. The second connector 30 is positioned to reversibly and adjustably couple to the first connector 26 to adjustably couple the second edge 32 of the panel 24 proximate to the first edge 28. The sleeve 12 is adjustably formed around the palm and the forearm of the user.

In one embodiment, the first connector 26 comprises a plurality of first straps 34 that is coupled to and extends from the panel 24. Each first strap 34 comprises a first fastener 36 that is coupled to and is positioned on an upper face 40 of the first strap 34.

A second fastener 38 is coupled to and is positioned a back face 42 of the sleeve 12. The second fastener 38 is complementary to the first fastener 36. In this embodiment, the second connector 30 comprises a plurality of rings 44 that is pivotally coupled to the panel 24. The rings 44 are rectangularly shaped. Each ring 44 is positioned to insert an associated first strap 34. The first fastener 36 is positioned to couple to the second fastener 38. The sleeve 12 is adjustably formed around the palm and the forearm of the user. In another embodiment, each first fastener 36 and an associated second fastener 38 comprises a first hook and loop fastener 46. In yet another embodiment, the plurality of first straps 34 comprises three first straps 34.

A hole 48 is positioned in the sleeve 12 proximate to the first end 14. The hole 48 is configured to insert a thumb of a hand of the user as the hand is inserted through the second end 16 of the sleeve 12 to position the thumb external to the sleeve 12.

The device 10 comprises a tube 50 that is configured to insert at least a portion of a distal phalanx of the thumb. A first coupler 52 and a second coupler 66 are coupled to the tube 50. The first coupler 52 is selectively couplable a side 54 of the sleeve 12. The second coupler 66 is selectively couplable to a palm face 68 of the sleeve 12. The first coupler 52 and the second coupler 66 are configured to adjustably substantially immobilize the thumb relative the hand of the user.

In one embodiment, the first coupler 52 comprises a second strap 56 and a third connector 58. The second strap 56 is coupled to and extends from a first endpoint 60 of the tube 50. The second strap 56 is flexible. The third connector 58 is coupled to the second strap 56 distal from the tube 50. In this embodiment, a fourth connector 62 is coupled to the side 54 of the sleeve 12. The fourth connector 62 is complementary to the third connector 58. The fourth connector 62 is positioned to reversibly and adjustably couple to the third connector 58 to adjustably couple the tube 50 to the side 54 of the sleeve 12. In another embodiment, the fourth connector 62 covers the side 54 of the sleeve 12. The third connector 58 is adjustably positionable to couple to the fourth connector 62. In yet another embodiment, the fourth connector 62 and the third connector 58 comprises a second hook and loop fastener 64.

In one embodiment, the second coupler 66 comprises a cord 70, a tab 72 and a fifth connector 74. The cord 70 has a first terminus 76 that is coupled to the tube 50. The cord 70 extends from the tube 50 through a first penetration 78 that is positioned through the outer layer 20 of the sleeve 12. The first penetration 78 is positioned proximate to the hole 48. The cord 70 extends through the sleeve 12 such that a second terminus 80 of the cord 70 protrudes from a second penetration 82 that is positioned through the outer layer 20 of the sleeve 12. The second penetration 82 is positioned proximate to the second end 16 of the sleeve 12. The tab 72 is coupled to the second terminus 80 of the cord 70. The fifth connector 74 is coupled to the tab 72. In this embodiment, a sixth connector 84 is coupled to the palm face 68 of the sleeve 12. The sixth connector 84 is complementary to the fifth connector 74. The sixth connector 84 is positioned to reversibly and adjustably couple to the fifth connector 74 to adjustably couple the tube 50 to the palm face 68 of the sleeve 12. In another embodiment, the sixth connector 84 substantially covers the palm face 68. The fifth connector 74 is adjustably positionable to couple to the sixth connector 84. In yet another embodiment, the sixth connector 84 and the fifth connector 74 comprises a third hook and loop fastener 86. In still yet another embodiment, the tab 72 is substantially circularly shaped.

In one embodiment, each of a pair of orifices 88 is adjacently positioned through the tube 50. A separation 90 is positioned through the tube 50. The separation 90 is positioned between the orifices 88. The cord 70 is positioned to insert through the orifices 88 to position the cord 70 for knotting to couple the cord 70 to the tube 50.

In another embodiment, a third penetration 92 is positioned through the outer layer 20 of the sleeve 12. The third penetration 92 is positioned between the first penetration 78 and the first end 14 of the sleeve 12. The third penetration 92 is positioned to selectively insert the cord 70. The tube 50 is selectively oriented relative to the sleeve 12.

A slat 94 is coupled to the palm face 68 of the sleeve 12. The slat 94 is rigid. The slat 94 extends from proximate to the first end 14 to proximate to the second end 16. The slat 94 is configured to substantially immobilize a wrist of the user. In one embodiment, the slat 94 is fixedly positioned between the inner layer 18 and the outer layer 20. In another embodiment, the slat 94 comprises aluminum.

In use, the hole 48 is configured to insert the thumb of the hand of the user. The rings 44 are positioned to insert associated first straps 34 such that the first fasteners 36 are positioned to couple to associated second fasteners 38. The sleeve 12 is adjustably formed around the palm and the forearm of the user and the thumb is positioned external to the sleeve 12. The slat 94 is configured to substantially immobilize the wrist of the user. The fourth connector 62 is positioned to reversibly and adjustably couple to the third connector 58 to adjustably couple the tube 50 to the side 54 of the sleeve 12. The sixth connector 84 is positioned to reversibly and adjustably couple to the fifth connector 74 to adjustably couple the tube 50 to the palm face 68 of the sleeve 12. The thumb is adjustably substantially immobilized relative the hand of the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A thumb rehabilitation splinting device comprising:
a sleeve, said sleeve having a first end and a second end, said first end and said second end being open, said sleeve being substantially complementary to a palm and a forearm of a user;
a hole positioned in said sleeve proximate to said first end;
a tube configured for inserting at least a portion of a distal phalanx of a thumb;
a first coupler coupled to said tube, said first coupler being selectively couplable to a side of said sleeve;
a second coupler coupled to said tube, said second coupler being selectively couplable to a palm face of said sleeve;
a slat coupled to said palm face of said sleeve, said slat being rigid, said slat extending from proximate to said first end to proximate to said second end; and
wherein said hole is positioned in said sleeve such that said hole is configured for inserting the thumb of a hand of the user as the hand is inserted through said second end of said sleeve positioning the thumb external to said sleeve, wherein said slat is positioned on said sleeve such that said slat is configured for substantially immobilizing a wrist of the user, wherein said first coupler and said second coupler are positioned on said tube such that said first coupler and said second coupler are configured for adjustably substantially immobilizing the thumb relative the hand of the user.

2. The device of claim 1, further including said sleeve comprising an inner layer and an outer layer.

3. The device of claim 1, further comprising:
a slit positioned in said sleeve, said slit extending from said first end to said second end such that said sleeve is selectively configurable as a panel;
a first connector coupled to a first edge of said panel;
a second connector coupled to a second edge of said panel, said second connector being complementary to said first connector; and
wherein said second connector is positioned on said panel such that said second connector is positioned for reversibly and adjustably coupling to said first connector for adjustably coupling said second edge of said panel proximate to said first edge such that said sleeve is adjustably formed around the palm and the forearm of the user.

4. The device of claim 3, further comprising:
said first connector comprising a plurality of first straps coupled to and extending from said panel, each said first strap having first fastener coupled to and positioned on an upper face of said first strap;
a second fastener coupled to and positioned on a back face of said sleeve, said second fastener being complementary to said first fastener
said second connector comprising a plurality of rings pivotally coupled to said panel, said rings being rectangularly shaped; and
wherein each said ring is positioned on said panel such that said ring is positioned for inserting an associated said first strap such that said first fastener is positioned for coupling to said second fastener such that said sleeve is adjustably formed around the palm and the forearm of the user.

5. The device of claim 4, further including said plurality of first straps comprising three said first straps.

6. The device of claim 4, further including each said first fastener and an associated said second fastener comprising a first hook and loop fastener.

7. The device of claim 1, further comprising:
said first coupler comprising a second strap and a third connector, said second strap being coupled to and extending from a first endpoint of said tube, said second strap being flexible, said third connector being coupled to said second strap distal from said tube;
a fourth connector coupled to said side of said sleeve, said fourth connector being complementary to said third connector; and
wherein said fourth connector is positioned on said side such that said fourth connector is positioned for reversibly and adjustably coupling to said third connector for adjustably coupling said tube to said side of said sleeve.

7. The device of claim 6, further including said fourth connector covering said side such that said third connector is adjustably positionable for coupling to said fourth connector.

8. The device of claim 6, further including said fourth connector and said third connector comprising a second hook and loop fastener.

9. The device of claim 2, further comprising:
said second coupler comprising a cord, a tab and a fifth connector, said cord having a first terminus coupled to said tube, said cord extending from said tube through a first penetration positioned through said outer layer of said sleeve, said first penetration being positioned proximate to said hole, said cord extending through said sleeve such that a second terminus of said cord protrudes from a second penetration positioned through said outer layer of said sleeve, said second penetration being positioned proximate to said second end of said sleeve, said tab being coupled to said second terminus of said cord, said fifth connector being coupled to said tab;
a sixth connector coupled to said palm face of said sleeve, said sixth connector being complementary to said fifth connector; and
wherein said sixth connector is positioned on said palm face such that said sixth connector is positioned for reversibly and adjustably coupling to said fifth connector for adjustably coupling said tube to said palm face of said sleeve.

10. The device of claim 9, further including said sixth connector substantially covering said palm face such that said fifth connector is adjustably positionable for coupling to said sixth connector.

11. The device of claim 9, further including said sixth connector and said fifth connector comprising a third hook and loop fastener.

12. The device of claim 9, further including said tab being substantially circularly shaped.

13. The device of claim 9, further including comprising:
a pair of orifices adjacently positioned through said tube;
a separation positioned through said tube, said separation being positioned between said orifices; and
wherein said orifices are positioned in said tube such that said cord is positioned for inserting through said orifices positioning said cord for knotting to couple said cord to said tube.

14. The device of claim 9, further including a third penetration positioned through said outer layer of said sleeve, said third penetration being positioned between said first penetration and said first end of said sleeve, wherein said third penetration is positioned in said sleeve such that said third penetration is positioned for selectively inserting said cord such that said that said tube is selectively oriented relative to said sleeve.

15. The device of claim 2, further including said slat being fixedly positioned between said inner layer and said outer layer.

16. The device of claim 1, further including said slat comprising aluminum.

17. A thumb rehabilitation splinting device comprising:
a sleeve, said sleeve having a first end and a second end, said first end and said second end being open, said sleeve being substantially complementary to a palm and a forearm of a user, said sleeve comprising an inner layer and an outer layer;
a slit positioned in said sleeve, said slit extending from said first end to said second end such that said sleeve is selectively configurable as a panel;
a first connector coupled to a first edge of said panel, said first connector comprising a plurality of first straps coupled to and extending from said panel, each said first strap having first fastener coupled to and positioned on an upper face of said first strap, said plurality of first straps comprising three said first straps;
a second fastener coupled to and positioned on a back face of said sleeve, said second fastener being complementary to said first fastener, each said first fastener and an associated said second fastener comprising a first hook and loop fastener;
a second connector coupled to a second edge of said panel, said second connector being complementary to said first connector, wherein said second connector is positioned on said panel such that said second connector is positioned for reversibly and adjustably coupling to said first connector for adjustably coupling said second edge of said panel proximate to said first edge such that said sleeve is adjustably formed around the palm and the forearm of the user, said second connector comprising a plurality of rings pivotally coupled to said panel, said rings being rectangularly shaped, wherein each said ring is positioned on said panel such that said ring is positioned for inserting an associated said first strap such that said first fastener is positioned for coupling to said second fastener such that said sleeve is adjustably formed around the palm and the forearm of the user;
a hole positioned in said sleeve proximate to said first end, wherein said hole is positioned in said sleeve, wherein said hole is positioned in said sleeve such that said hole is configured for inserting a thumb of a hand of the user as the hand is inserted through said second end of said sleeve positioning the thumb external to said sleeve;
a tube configured for inserting at least a portion of a distal phalanx of the thumb,
a first coupler coupled to said tube, said first coupler being selectively couplable a side of said sleeve, said first coupler comprising a second strap and a third connector, said second strap being coupled to and extending from a first endpoint of said tube, said second strap being flexible, said third connector being coupled to said second strap distal from said tube;
a fourth connector coupled to said side of said sleeve, said fourth connector being complementary to said third connector, wherein said fourth connector is positioned on said side such that said fourth connector is positioned for reversibly and adjustably coupling to said third connector for adjustably coupling said tube to said side of said sleeve, said fourth connector covering said side such that said third connector is adjustably positionable for coupling to said fourth connector, said fourth connector and said third connector comprising a second hook and loop fastener;
a second coupler coupled to said tube, said second coupler being selectively couplable to a palm face of said sleeve, wherein said first coupler and said second coupler are positioned on said tube such that said first coupler and said second coupler are configured for adjustably substantially immobilizing the thumb relative the hand of the user, said second coupler comprising a cord, a tab and a fifth connector, said cord having a first terminus coupled to said tube, said cord extending from said tube through a first penetration positioned through said outer layer of said sleeve, said first penetration being positioned proximate to said hole, said cord extending through said sleeve such that a second terminus of said cord protrudes from a second penetration positioned through said outer layer of said sleeve, said second penetration being positioned proximate to said second end of said sleeve, said tab being coupled to said second terminus of said cord, said fifth connector being coupled to said tab, said tab being substantially circularly shaped;
a pair of orifices adjacently positioned through said tube;
a separation positioned through said tube, said separation being positioned between said orifices, wherein said orifices are positioned in said tube such that said cord is positioned for inserting through said orifices positioning said cord for knotting to couple said cord to said tube;
a third penetration positioned through said outer layer of said sleeve, said third penetration being positioned between said first penetration and said first end of said sleeve, wherein said third penetration is positioned in said sleeve such that said third penetration is positioned for selectively inserting said cord such that said that said tube is selectively oriented relative to said sleeve;
a sixth connector coupled to said palm face of said sleeve, said sixth connector being complementary to said fifth connector, wherein said sixth connector is positioned on said palm face such that said sixth connector is positioned for reversibly and adjustably coupling to said fifth connector for adjustably coupling said tube to said palm face of said sleeve, said sixth connector substantially covering said palm face such that said fifth connector is adjustably positionable for coupling to said sixth connector, said sixth connector and said fifth connector comprising a third hook and loop fastener;
a slat coupled to said palm face of said sleeve, said slat being rigid, said slat extending from proximate to said first end to proximate to said second end, wherein said slat is positioned on said sleeve such that said slat is configured for substantially immobilizing a wrist of the user, said slat being fixedly positioned between said inner layer and said outer layer, said slat comprising aluminum; and
wherein said hole is positioned in said panel such that said hole is configured for inserting the thumb of the hand of the user, wherein each said ring is positioned on said panel such that said ring is positioned for inserting an associated said first strap such that said first fastener is positioned for coupling to said second fastener such that said sleeve is adjustably formed around the palm and the forearm of the user and positioning the thumb external to said sleeve, wherein said slat is positioned on said sleeve such that said slat is configured for substantially immobilizing the wrist of the user, wherein said fourth connector is positioned on said side such that said fourth connector is positioned for reversibly and adjustably coupling to said third connector for adjustably coupling said tube to said side of said sleeve, wherein said sixth connector is positioned on said palm face such that said sixth connector is positioned for reversibly and adjustably coupling to said fifth connector for adjustably coupling said tube to said palm face of said sleeve for adjustably substantially immobilizing the thumb relative the hand of the user.
